(19) United States
(12) Patent Application Publication
(10) Pub. No.: US 2014/0155693 A1
(43) Pub. Date: Jun. 5, 2014

(54) ENDOSCOPIC DELIVERY SYSTEM

(71) Applicants: Izhak Fabian, Kfar Truman (IL); Nir Altman, Kibbutz Kfar Etzion (IL); Steven Haas, Kochav Yair (IL); Yoav Hirsch, Modiin (IL)

(72) Inventors: Izhak Fabian, Kfar Truman (IL); Nir Altman, Kibbutz Kfar Etzion (IL); Steven Haas, Kochav Yair (IL); Yoav Hirsch, Modiin (IL)

(21) Appl. No.: 13/705,369

(22) Filed: Dec. 5, 2012

(51) Int. Cl. A61B 1/012; A61B 1/00

(52) U.S. Cl. CPC A61B 1/0125; A61B 1/00131
USPC 600/106; 600/104

(57) ABSTRACT

An endoscopic delivery system including an endoscope including at least one imaging device and a working channel, and a catheter arranged to pass through the working channel, wherein the catheter is formed with an observation window on a side portion thereof, the window being in a line of sight of the at least one imaging device.

### FIELD OF THE INVENTION

The present invention generally relates to endoscopic delivery devices, in particular for delivering medical devices across a barrier.

### BACKGROUND OF THE INVENTION

In the prior art, when an occlusion of the pylorus is required in the course of a gastroplasty procedure or in a procedure that involves the duodenum, the surgeon staples the pylorus shut (in the stomach) and this is a short term occlusion to allow the duodenum to recover from an operation. Transpyloric devices have also been proposed, which may partially and/or intermittently obstruct the pylorus, thereby decreasing the flow of gastric contents into the duodenum.

However, endoscopic delivery of pylorus devices (such as a pylorus plug) is complicated by the fact that the distal portion of the device, after passing through the pylorus, is not viewable by the endoscope.

### SUMMARY OF THE INVENTION

The present invention seeks to provide an improved endoscopic delivery device, particularly useful for viewing delivery of a medical device through an opening in a barrier (such as, but not limited to, delivery of a pylorus plug across the pylorus), as is described more in detail hereinbelow. The device is particularly useful in a transoral gastrointestinal procedure, but the invention is not limited to transoral gastroplasty, and may be used in other laparoscopic, endoscopic, or natural orifice procedures in other body lumens.

There is thus provided in accordance with an embodiment of the present invention an endoscopic delivery system including an endoscope including at least one imaging device and a working channel, and a catheter arranged to pass through the working channel, wherein the catheter is formed with an observation window on a side portion thereof, the window being in a line of sight of the at least one imaging device.

In accordance with an embodiment of the present invention the at least one imaging device is positioned on a distal face of the endoscope to have a front field of view. A grasping tool may be used to grasp a medical device disposed in the catheter.

In accordance with an embodiment of the present invention the grasping tool includes an advancement indicator at a proximal end thereof, such as markings and/or a physical click mechanism.

There is also provided in accordance with an embodiment of the present invention an endoscopic method including employing an endoscope that includes at least one imaging device and a working channel, passing a catheter through the working channel, the catheter being formed with an observation window on a side portion thereof, and the window being in a line of sight of the at least one imaging device, delivering a medical device (e.g., a pylorus plug) through the catheter, and viewing advancement of the medical device with respect to the catheter by using the at least one imaging device to view the advancement through the window.

In accordance with an embodiment of the present invention a non-distal portion of the medical device is viewed via the window without having to view a distal tip of the medical device.

In accordance with an embodiment of the present invention a grasping tool is used to grasp the medical device, and the grasping tool is used both to deliver and retrieve the medical device.

In accordance with an embodiment of the present invention the medical device is delivered through an opening in a barrier, wherein a distal portion of the medical device, after passing through the barrier, is not viewable by the at least one imaging device.

### BRIEF DESCRIPTION OF THE DRAWINGS

The present invention will be understood and appreciated more fully from the following detailed description taken in conjunction with the drawings in which:

FIGS. 1A and 1B are simplified pictorial illustrations of an endoscopic delivery system, respectively before and after delivery of a medical device, constructed and operative in accordance with an embodiment of the present invention.

### DETAILED DESCRIPTION OF EMBODIMENTS

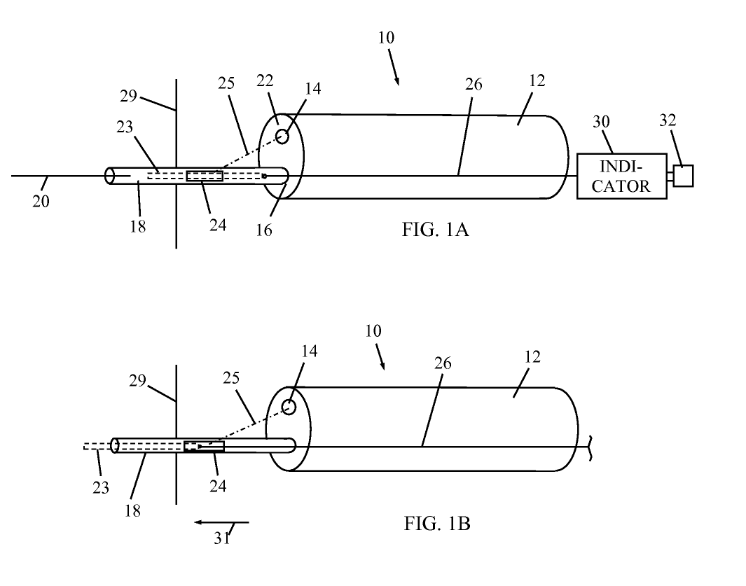

Reference is now made to FIGS. 1A-1B, which illustrate an endoscopic delivery system 10, constructed and operative in accordance with an embodiment of the present invention.

System 10 includes an endoscope 12 that has at least one imaging device 14, such as a CCD, and a working channel 16. The working channel 16 may be used to pass therethrough a catheter (or cannula) 18, which may go over or through a guidewire 20 (shown in FIG. 1A only). The imaging device 14 is positioned on a distal face 22 of endoscope 12, to have a direct, front field of view.

A medical device 23, such as a balloon, stent, plug and others, may be delivered through catheter 18. Catheter 18 is formed with an observation window 24 on a side portion thereof. Window 24 enables viewing the advancement of device 23 with respect to catheter 18, such as by means of imaging device 14 (window 24 is in its line of sight, as indicated by the broken line 25). For example, FIG. 1A illustrates introducing device 23 through catheter 18 by means of a grasping tool 26. In FIG. 1A, a middle portion of device 23 is seen through window 24. In FIG. 1B, it can be seen that device 23 has been advanced sufficiently (in the direction of an arrow 31) that the proximal end of device 23 is seen through window 24. Observation window 24 thus enables the operator to know device 23 has reached its intended destination in a body lumen without having to image the distal tip of device 23 or catheter 18. This may be particular helpful if device 23 must pass through an opening in a barrier 29, such as but not limited to, the pylorus in the gastrointestinal tract. This may be helpful in delivering a pylorus plug or other medical device 23, for example. In such an example, after advancing catheter 18 through the pylorus (barrier 29), imaging device 14 cannot view the plug (device 23) exiting the distal end of catheter 18, but can view the progress through window 24. During retrieval of catheter 18 in the proximal direction, imaging device 14 can view, via window 24, how far the proximal side of the plug was advanced.

The grasping tool 26 may include an advancement indicator 30 at a proximal end thereof (such as near a proximal handle 32). The advancement indicator 30 may include markings or a physical click mechanism that measures the delivery distance of medical device 23 through catheter 18. Indicator 30 also indicates when catheter 18 can be pulled back proximally after delivery of device 23 to its desired location.

The endoscopic delivery system 10, with grasping tool 26, can be used for both insertion and retrieval of medical device 23.

It will be appreciated by persons skilled in the art that the present invention is not limited by what has been particularly shown and described hereinabove. Rather the scope of the present invention includes both combinations and subcombinations of the features described hereinabove as well as modifications and variations thereof which would occur to a person of skill in the art upon reading the foregoing description and which are not in the prior art.

What is claimed is:

1. An endoscopic delivery system comprising:
an endoscope comprising at least one imaging device and a working channel; and
a catheter arranged to pass through said working channel, wherein said catheter is formed with an observation window on a side portion thereof, said window being in a line of sight of said at least one imaging device.

2. The system according to claim 1, wherein said at least one imaging device is positioned on a distal face of said endoscope to have a front field of view.

3. The system according to claim 1, further comprising a grasping tool operative to grasp a medical device disposed in said catheter.

4. The system according to claim 3, wherein said grasping tool comprises an advancement indicator at a proximal end thereof.

5. The system according to claim 4, wherein said advancement indicator comprises markings.

6. The system according to claim 4, wherein said advancement indicator comprises a physical click mechanism.

7. An endoscopic method comprising:
employing an endoscope that comprises at least one imaging device and a working channel;
passing a catheter through said working channel, said catheter being formed with an observation window on a side portion thereof, and said window being in a line of sight of said at least one imaging device;
delivering a medical device through said catheter; and
viewing advancement of said medical device with respect to said catheter by using said at least one imaging device to view the advancement through said window.

8. The method according to claim 7, wherein viewing advancement of said medical device comprises viewing a non-distal portion of said medical device via said window without having to view a distal tip of said medical device.

9. The method according to claim 7, wherein a grasping tool is used to grasp said medical device, and said grasping tool is used both to deliver and retrieve said medical device.

10. The method according to claim 7, comprising delivering said medical device through an opening in a barrier, wherein a distal portion of said medical device, after passing through said barrier, is not viewable by said at least one imaging device.

11. The method according to claim 7, wherein said medical device comprises a pylorus plug.
